(19) United States
(12) Patent Application Publication
(10) Pub. No.: US 2013/0289138 A1
(43) Pub. Date: Oct. 31, 2013

(54) COMPOSITE MATERIAL SUITABLE FOR BIOMEDICAL USE, AND METHOD TO PRODUCE IT

(71) Applicants: Francesco Segatti, Udine (IT); David Mercuri, Udine (IT)

(72) Inventors: Francesco Segatti, Udine (IT); David Mercuri, Udine (IT)

(73) Assignee: LIMACORPORATE SPA, San Daniele del Friuli (IT)

(21) Appl. No.: 13/996,205

(22) PCT Filed: Dec. 21, 2011
(86) PCT No.: PCT/IB11/03102

§ 371 Date: Jun. 20, 2013

(30) Foreign Application Priority Data
Dec. 22, 2010 (IT) UD2010A000233

(51) Int. Cl. A61K 9/00

(52) U.S. Cl. CPC A61K 9/0024
USPC 514/778; 514/779; 514/781

(57) ABSTRACT

Composite material suitable for biomedical use, comprising a first phase consisting of a reticulated hydrogel deriving from a polymer chosen from among polymers N-methyl-amides derivatives of carboxymethylcellulose, alginic acid or carboxymethyl starch and a second phase which comprises a solution of the above polymers.

### FIELD OF THE INVENTION

The present invention concerns a composite material for biomedical uses, and the method to produce it, which provides to compatibilize a polymer fraction inside a three-dimensional reticulated matrix, such as gel.

For example, but not only, the material according to the present invention can be used as a biomedical device for treating osteoarthritis, in particular for the visco-supplementation of degenerated synovial liquid.

### BACKGROUND OF THE INVENTION

In the biomedical field, there is a need to develop new materials, in particular suitable for intra-articular application, which show a good compromise between elastic and viscous module. It is known that a biomedical material that has too high a value of elastic module is not suitable for application in the treatment of osteoarthritis, in particular for the visco-supplementation of degenerated synovial liquid, since it tends to fragment.

Moreover, if the biomedical material has too high a viscous module, it tends only to thicken the synovial liquid.

A versatile and frequently used biomaterial is hyaluronic acid, which is used in weakly reticulated preparations but which is expensive, rapidly degradable and with poor visco-elastic properties. Document EP-A-0.466.300 (EP'300) describes a biocompatible visco-elastic slurry gel, formed by a polymer gel with a hyaluronic acid base and a fluid phase formed by a water solution of a polymer, such as for example hyaluronic acid itself

Polymers N-methyl-amides derivatives of carboxymethylcellulose, alginic acid or carboxymethyl starch, used in the biomedical field, are also known, from the European patent EP-B-1.614.696 (EP'696), in the name of the present Applicant.

These polymers, which have a semi-synthetic derivation, not animal or biotechnological, do not have any protein contaminants. However, neither EP'696 nor EP'300 supply any teaching on the rheological behavior of the polymers described therein, either reticulated or in solution.

Purpose of the present invention is to produce a material, and to perfect a relative method to produce it, which has a good compromise between elastic module and viscous module, so that it can be used in biomedical applications, in particular intra-articular.

The Applicant has devised, tested and embodied the present invention to overcome the shortcomings of the state of the art and to obtain these and other purposes and advantages.

Unless otherwise defined, all the technical and scientific terms used here and hereafter have the same meaning as commonly understood by a person with ordinary experience in the field of the art to which the present invention belongs. Even if methods and materials similar or equivalent to those described here can be used in practice and in the trials of the present invention, the methods and materials are described hereafter as an example. In the event of conflict, the present application shall prevail, including its definitions. The materials, methods and examples have a purely illustrative purpose and shall not be understood restrictively.

The word “comprise” and variants of the word such as “comprises” and “comprising” are used here to indicate the inclusion of a clearly expressed whole or clearly expressed wholes but not the exclusion of any other whole or any other wholes, unless in the context or in use an exclusive interpretation of the word is required.

### SUMMARY OF THE INVENTION

The present invention is set forth and characterized in the independent claims, while the dependent claims describe other characteristics of the invention or variants to the main inventive idea.

The present invention is based on what is described in EP'696, which is entirely incorporated here for reference.

In particular, the present invention uses the polymers N-methyl-amides derivatives of carboxymethylcellulose, alginic acid or carboxymethyl starch as in EP'696 as a starting product to achieve a composite material suitable for biomedical use, in particular for use in the treatment of osteoarthritis, even more particularly for the visco-supplementation of degenerated synovial liquid.

Following experiments carried out by Applicant, the products have shown unexpected chemical-physical, rheological and biological properties that satisfy the following requirements:

their behavior is reproducible and modulatable in terms of viscosity, swelling and visco-elasticity of water solutions/dispersions, comparable with that of hyaluronic acid or its functionalized and/or reticulated derivatives;

they can be worked in various forms, such as filaments, tissues, spreadable or injectable gel;

easily sterilized in the form of raw products, semi-worked or finished;

low cost of the original materials and derivatives, including the reticulates, therefore making the products suitable for use in the field of foodstuffs and pharmaceuticals, the medical and cosmetic fields. The composite material according to the present invention comprises a first phase consisting of a three-dimensional matrix of a reticulated hydrogel deriving from one of the polymers N-methyl-amides derivatives of carboxymethylcellulose, alginic acid or carboxymethyl starch, and a second phase that comprises one of said polymers.

In particular, the material according to the present invention is obtained starting from a dispersion of highly reticulated hydrogel deriving from one of the above polymers in one of the above polymers, for example in powder form of one of the above polymers, or in a solution, in which the solvent is mainly water, of one of the above polymers.

In a first form of embodiment, the reticulated hydrogel is in dried powder form.

In a second form of embodiment, the reticulated hydrogel is in solution.

In some forms of embodiment, the degree of occupation of the carboxyl functions of the hydrogel is advantageously greater than about 70%, that is, up to 50% amidation of the polymer chains and at least 20% of reticulation of the functional groups of the polymer that are still free.

In one form of embodiment, in the composite material according to the present invention there are from about 2 to about 5, preferably from about 2.5 to about 4.5, even more preferably from about 3 to about 4 parts of reticulated hydrogel (first phase) and from about 5 to about 8, preferably from about 5.5 to about 7.5, even more preferably from about 6 to about 7 parts of polymer as such (second phase), with the provision that the sum of the parts of the two phases, on a weight basis, is equal to 10.

Some forms of embodiment provide that the ratio between the first phase and the second phase, on a weight basis, in the composite material according to the present invention, can be: 2:8, 2.5:7.5, 3:7, 3.5:6.5, 4:6, 4.5:5.5, 5:5.

In particular, in the above polymers the percentage of N-methyl-amide of the original carboxyl groups in the polysaccharide (AD, substitution or amidation degree) is less than 100%.

Preferably, the same polymer is used for both the first phase and the second phase which form the composite material, selecting on each occasion, among the N-methyl-amide of carboxymethylcellulose, alginic acid or carboxymethyl starch, the polymer suitable to make the desired biomedical device.

The method according to the present invention provides that one of the polymers is prepared and purified, obtaining a polymer solution, for example according to one of the methods described in EP'696, or by means of other purification techniques, such as tangential filtration. Purification is advantageously greater than 99%.

Advantageously, the preparation and purification performed provides to obtain a polymer solution that contains from about 1% to about 2% in weight, preferably from about 1.25% to about 1.75% in weight, of a selected one of the polymers.

This concentration of the polymer solution is advantageous because it represents a compatible material useful for obtaining a viscous module greater than 75 Pa (2.5 Hz) compared with a lower enzymatic degradability, for example Hyaluronidase (Type 1), if compared with an analogous solution of hyaluronic acid.

Subsequently, according to a first form of embodiment of the method according to the present invention, the polymer solution is concentrated by means of partial dehydration.

In this first form of embodiment, a first fraction or part of the concentrated polymer solution is subjected to an operation in which the solvent is totally removed by means of a drying technique, for example freeze-drying, and then subjected to grinding and sieving in order to obtain particles of dried powder that have a homogeneous distribution of the nominal diameter, preferably a nominal diameter less than or equal to about 2 mm, for example 1 mm, 1 5 mm or 2 mm, to prepare the first phase.

A second fraction or part of the concentrated polymer solution is subjected to reticulation, for example according to one of the methods described in EP'696, obtaining the reticulated hydrogel, which is subsequently purified by washing, in this case too, for example, as described in EP'696, or using other alternative techniques.

In a first variant, the reticulated hydrogel is then concentrated and the washing liquid used in the purification is totally removed, for example by freeze-drying.

Then the reticulated and concentrated hydrogel is subjected to grinding and and sieving in order to obtain particles of dried powder that have a homogeneous distribution of the nominal diameter in order to obtain said second phase, preferably a nominal diameter less than or equal to about 2 mm, for example 1 mm, 1.5 mm or 2 mm.

In particular, in this variant form of embodiment, for the purposes of biomedical use, a desired quantity of the powdered polymer material thus obtained, and a desired quantity of the powdered gel material are hydrated in a physiological solution, or sterile water.

In a second variant of the first form of embodiment of the present invention it is possible to disperse a desired quantity of powdered gel in the polymer solution previously prepared. In this second variant form of embodiment, the particles of hydrogel are dispersed in the polymer solution. In particular, in this form of embodiment, the dehydrated hydrogel is put into contact with the polymer solution, thus enabling the polymer to penetrate inside the three-dimensional reticular structure of the hydrogel. The process can also occur dynamically, for example using a closed rotating system (dynamic compatibilization). The re-hydration of the hydrogel by means of the polymer solution allows the chains to migrate inside the lattice.

The first and second variant of the first form of embodiment have the advantage that they allow an exact control of the relative quantities of the two components and hence to obtain a highly homogeneous final product.

In a second form of embodiment of the present invention the purified gel, still in solution, is compatibilized with the polymer solution so that the polymer penetrates inside the three-dimensional reticular structure of the hydrogel. The process occurs dynamically, for example using a closed rotating system (dynamic compatibilization). The re-hydration of the hydrogel and the polymer solution allows the chains to migrate inside the lattice.

Subsequently, in the second form of embodiment, the solvent is removed, for example by freeze-drying or drying, and the overall product obtained is ground and sieved so as to obtain the powdered particles with desired nominal sizes, preferably less than or equal to about 2 mm, for example 1 mm, 1.5 mm or 2 mm.

In particular, in the second form of embodiment, for biomedical use, a desired quantity of the powdered material thus obtained is dispersed in a physiological solution, or in sterile water, so that it can subsequently be injected.

In any case, for both forms of embodiment as described above, the nominal size of the particles obtained according to the present invention is selected as particularly suitable and specific for intra-articular injection.

The composite material according to the present invention, once suitably hydrated and packaged, can advantageously be subjected to sterilization, for example using steam.

The present invention provides an optimal compromise between the values of elastic module, to which the reticulated component of the material contributes, and which has the effect of dissipating the loads exerted during use on the joints, and of the viscous module, to which the polymer component of the material contributes.

The composite material obtained with the present invention has the property that it is visco-elastic in particular due to the effect of two factors:

the presence of fragments of hydrogel inside the solution to be injected confers consistency, mechanical resistance and ability to absorb shocks;

the polymer with high molecular weight incorporated inside the hydrogel increases the consistency of the hydrogel and increases the viscosity of the water.

Whether the two phases are ground and sieved independently of each other and compatibilized only at the end, or are compatibilized at the beginning by diffusion in a water environment and then subsequently ground and sieved together, the composite material obtained can be packaged and conserved in a package, such as an envelope, bag, syringe, phial or similar container for biomedical use, of the sterile type.

All in all, the final composite material contains, in a water or physiological solution suitable for supplementation for example by means of injection, from about 1.75% to about 5% in weight, preferably from about 2% to about 3.75% in weight, for example about 3.61%, of a selected one of the polymers and the reticulated hydrogel deriving from one of the polymers.

### EXAMPLE 1

A polymer N-methyl-amides derivatives of carboxymethylcellulose, alginic acid or carboxymethyl starch is produced as described in EP'696. The polymer produced is in a water solution, for example about 1.5% in weight.

Depending on whether the first or second form of embodiment of the present invention is used, the polymer solution can be concentrated, for example by means of a rotary drier.

According to the first variant of the first form of embodiment of the present invention, the solvent is totally removed from a first part of the concentrated polymer solution by means of drying, for example freeze-drying.

Furthermore, a second part of the concentrated polymer solution produced is subjected to reticulation, as described in EP'696, to obtain a hydrogel that is purified by means of successive washings in a saline solution 0.1M or in water. Alternatively, other purification techniques may be used.

Then the swelling water is totally removed from the hydrogel by means of a suitable process, such as drying and/or freeze-drying.

The two dried phases of polymer and reticulated hydrogel are subjected to grinding and sieving, separately from each other, so as to obtain powders of the two phases with a nominal diameter equal to about 2 mm. The two powders of the first and second phase can be dispersed separately from each other in a water or physiological solution and then compatibilized when they are introduced into the injection member.

On the contrary, in the second variant of the first form of embodiment of the present invention, the hydrogel in the form of solid powder obtained is put into contact with the polymer solution as previously prepared, obtaining the incorporation of the polymer in the lattice. The water present in the sample is then eliminated and when this operation is complete the product is ground so as to obtain a compound with homogeneous sizes and equal to about 2 mm nominal diameter.

Or, according to the second form of embodiment of the present invention, it is also possible to compatibilize the purified gel still in solution in the polymer solution as previously prepared.

The ratio between the reticulated hydrogel and the powder used is respectively 2 to 3. Another example of the ratio between the reticulated hydrogel and the polymer is 3 to 7.

### EXAMPLE 2

The powdered composite material in example 1 is dispersed in sterile water, for example about 150 mg of composite material in about 4 ml of solution. Once hydrated, the hydrogel swells, allowing the polymer to spread into the surrounding environment for the desired effect of intra-articular treatment.

### COMPARATIVE EXAMPLE 1

Applicant has carried out a rheological characterization of the material of the present invention, comparing it with a material formed completely by one of the polymers N-methyl-amides derivatives of carboxymethylcellulose, alginic acid or carboxymethyl starch (comparative sample 10, 100% polymer), and also with a material formed completely by a reticulated hydrogel deriving from said polymers (comparative sample 0, 100% reticulate).

Eleven samples were prepared in water solution, with a concentration of 3.61% w/w, containing different polymer/gel ratios. Sample zero (0) has a single gel component, sample one (1) has a polymer/gel ratio of 1/9 and so on, until sample ten (10) that has a polymer component only.

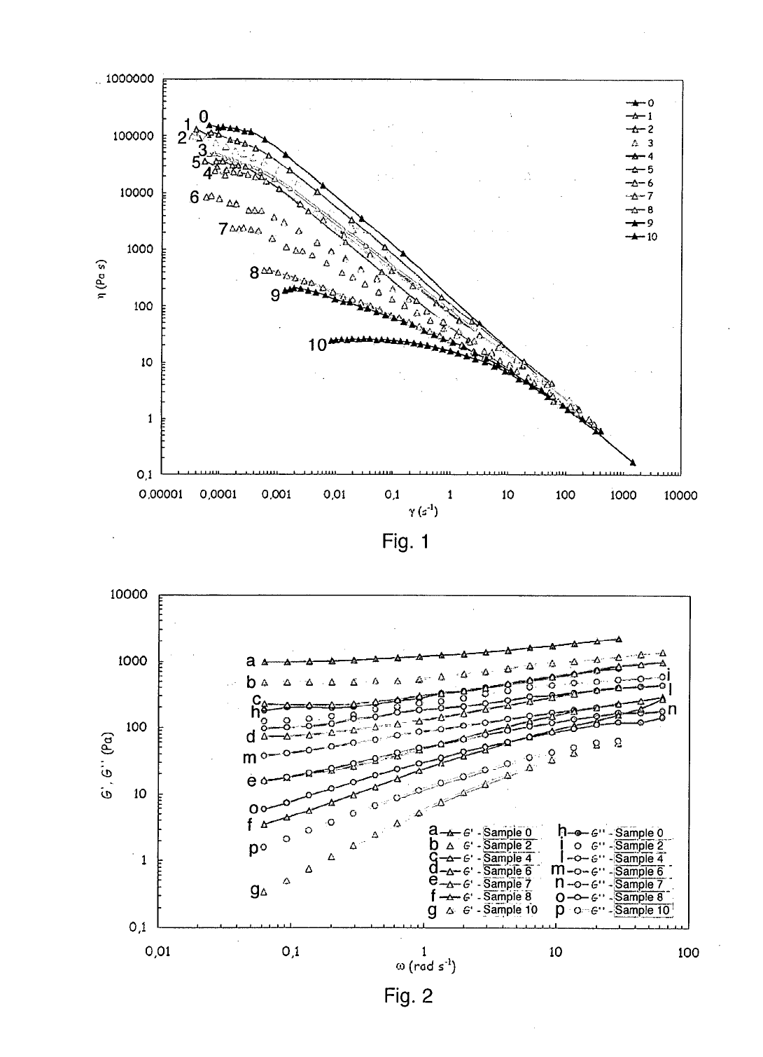

The results of the comparison are shown in FIG. 1, which shows a graph of the stationary viscosity (η [Pa*s] on the y-axis) as a function of the speed of deformation (γ [s⁻¹] on the x-axis), and in FIG. 2 which shows a graph of the mechanical spectra (ω [rad*s⁻¹] on the x-axis, G′, G″, [Pa] on the y-axis) for the eleven samples analyzed.

The comparative sample 10 showed a behavior substantially comparable to a non-Newtonian fluid, that is, the pseudo-plastic type.

The comparative sample 0 showed a high elastic module and low viscous module, with the disadvantages that that entails.

The sample according to the present invention, on the contrary, has a behavior similar to pseudo-plastic but with a good compromise between elastic and viscous module.

### COMPARATIVE EXAMPLE 2

Applicant has carried out a test, comparing the degradation of the hyaluronic acid polymer (Hyal) with a polymer according to the present invention, in this case a polymer N-methyl-amides derivatives of carboxymethylcellulose (CMC-A), by means of an enzyme present inside the human organism on an articular level, especially in the event of inflammation, for example Hyaluronidase (Type 1). The comparison was carried out on the polymeric component since, according to literature, it is known that the reticulated component reduces degradation times.

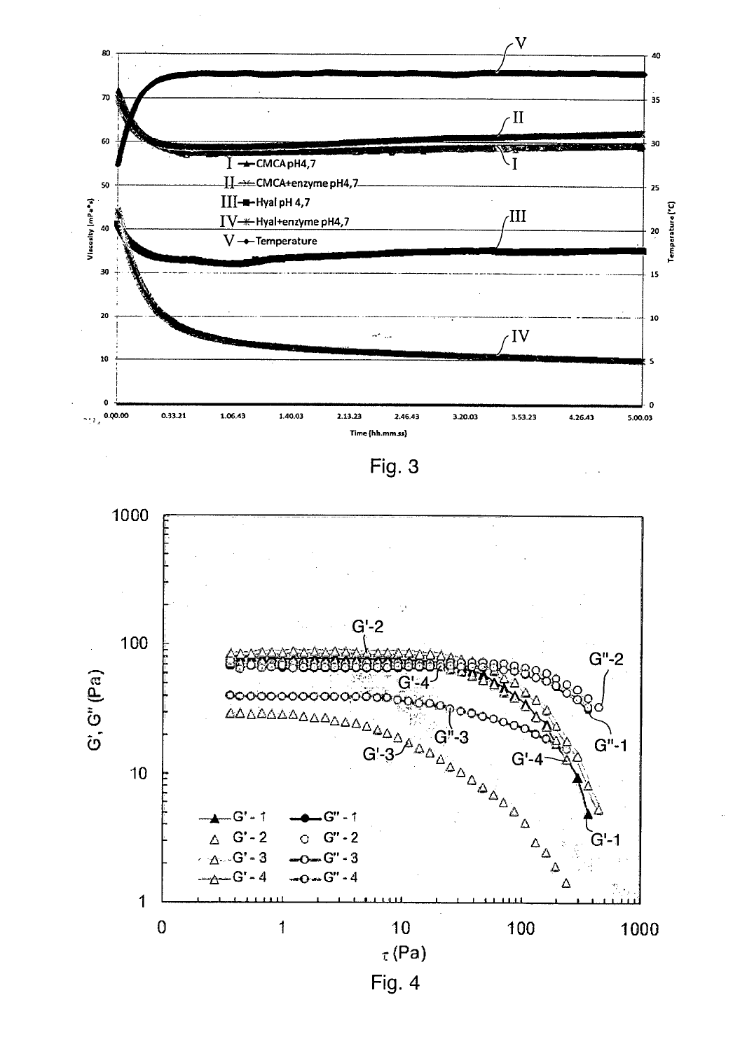

The test was carried out evaluating the viscosity of the solution over time, at a temperature of 37° C. and with a pH of 4.7, since the viscosity of the polymer depends on the molecular weight, and the enzyme acts by reducing the length of the polymeric chain and consequently the weight. The test was carried out for both types of polymer both with and without enzyme, in the same conditions, and the results obtained are shown in FIG. 3. While the hyaluronic acid, in the presence of the enzyme, shows a consistent loss of viscosity already after 30 minutes, equal to 50%, the polymer according to the present invention shows the same development both with and without the enzyme, in the same conditions, and therefore the enzyme has no degradation effects on the polymer in the time considered (and up to 24 hours).

### COMPARATIVE EXAMPLE 3

Applicant has carried out a test to compare the mechanical properties by means of a rheometric analysis of samples 1, 2, 3 and 4 of composite material according to the present invention, prepared as described in Example 1.

In particular, a first group of components 1, 2 and 4 was prepared according to the first form of embodiment of the present invention, using different techniques for grinding the reticulated gel.

A second component 3 was instead prepared according to the second form of embodiment of the present invention, that is, omitting the step of concentrating the hydrogel, for example by means of a rotary evaporator, carried out before freeze-drying.

This comparison emphasizes the advantages, for the purposes of the mechanical properties of the final product, of the operation of preparing the ground and sieved powder of gel which is compatibilized with polymer powder or polymer solution.

The differences between the samples emerge to a similar extent from the oscillatory tests, both from scanning the stresses and from scanning the frequencies, as can be seen in FIG. 4. The deviation from the linear viscoelastic behavior occurs with lower stresses in the case of sample 3. The mechanical profiles of the samples are similar to each other: the traces of the two modules are very close, and almost rectilinear in the region of the intermediate frequencies, whereas they diverge at lower frequencies, which indicates a visco-elastic behavior just beyond the sol-gel transition. Compared with the other samples, sample 3 is in a band of lower values of the two modules, since the difference is greater at low frequencies.

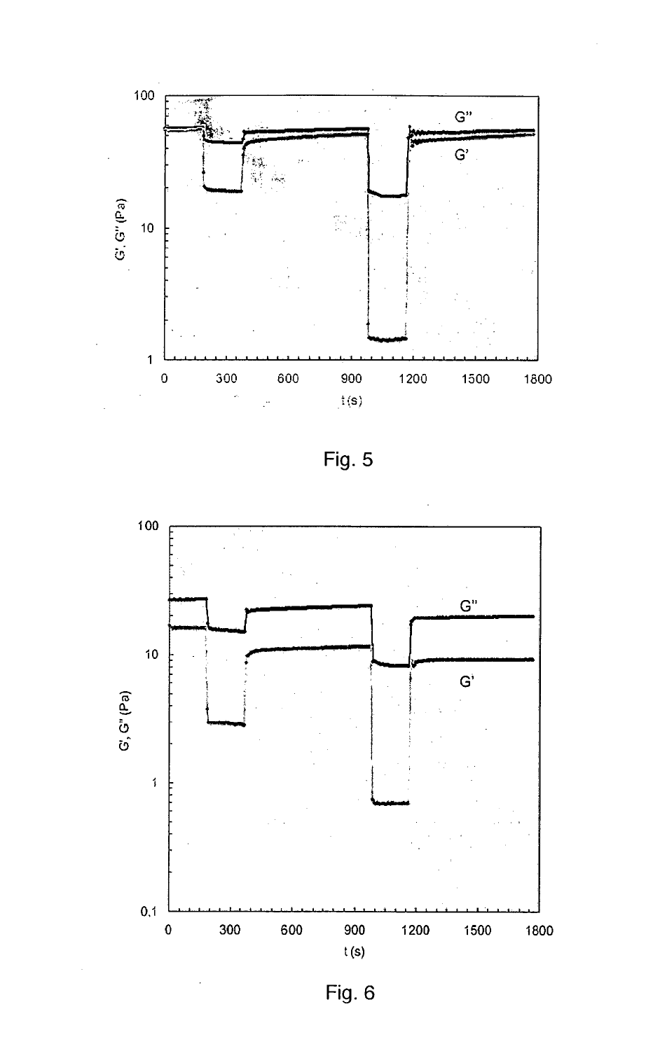

FIGS. 5 and 6 show the results of the recovery test carried out completely in conditions of oscillatory motion at fixed frequency (1 Hz), interposing segments with a greater oscillation amplitude (100 Pa and 500 Pa) than the reference segments at low stress (1 Pa). The results obtained for sample 1 are similar to those of samples 2 and 4, and differ from those of sample 3. In the first case, it can be observed that even when the oscillations with a very wide amplitude produce a substantial reduction of the modules, in particular of G′, there is a substantial recovery of both modules in the following segment. On the contrary, in the case of sample 3, the de-structuration is obvious and the recovery is less, in particular in the last segment.

A recovery test after a segment with a constant deformation speed (100 s⁻¹) was made on the standard system: 66% for G′ and 80% for G″, which percentage is drastically reduced when no concentration by drying is carried out on the gel.

### COMPARATIVE EXAMPLE 4

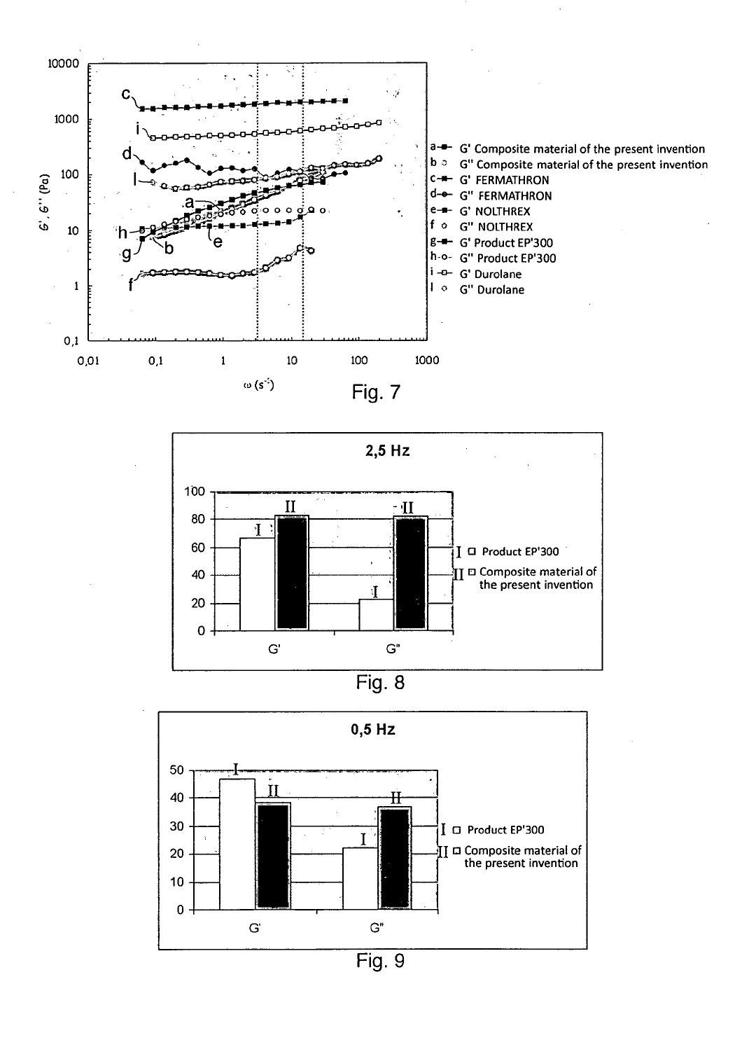

Applicant carried out a test to compare the mechanical characteristics by means of a rheometric analysis of the composite material prepared according to the first form of embodiment of the present invention, and some known products present on the market, including Fermathron, Nolthrex, Durolane and products deriving from EP'300 (FIG. 7).

Among the known products present on the market, products deriving from EP'300 and that developed by Applicant (whose G′ and G″ values are indicated by A and b in FIG. 7) are those in a central response band, supplying behaviors comparable with those of the synovial fluid.

Applicant also compared the rheological behavior of the product deriving from EP'300 with the one developed by Applicant (FIGS. 8 and 9).

From this comparison it emerges that there is a better performance in terms of visco-elasticity at high frequencies (quick walk/run) and a behavior substantially comparable to low frequencies (slow walk).

From the comparative examples reported above, it can be concluded that the material according to the present invention generally has the following advantageous properties:

i) elastic module G′ greater than 31 Pa and viscous module G″ greater than 32 Pa, recorded at a frequency of 0.5 Hz;
ii) elastic module G′ greater than 70 Pa and viscous module G″ greater than 70 Pa, recorded at a frequency of 2.5 Hz;
iii) recovery capacity after a segment with a constant speed of deformation (100 s−1): equal to 66% for G′ and 80% for G″.

All the results were recorded after injection of the product by means of a syringe with an 18G needle or in conditions similar to conditions of use.

1. Composite material suitable for biomedical use, comprising a first phase comprising a reticulated hydrogel deriving from a polymer chosen from among polymers N-methyl-amides derivatives of carboxymethylcellulose, alginic acid or carboxymethyl starch and a second phase which comprises one of said polymers.

2. Material as in claim 1, wherein the material comprises from about 2 to about 5 parts of reticulated hydrogel and from about 5 to about 8 parts of polymer, with the provision that the sum of the parts of the two phases, on a weight basis, is equal to 10.

3. Material as in claim 2, wherein a ratio between the first phase and the second phase, on a weight basis, is selected between: 2:8, 2.5:7.5, 3:7, 3.5:6.5, 4:6, 4.5:5.5, 5:5.

4. Material as in claim 1, wherein a degree of occupation of the carboxyl functions of the hydrogel is greater than about 70%.

5. Material as in claim 1, wherein the polymer of the second phase is the same reticulated polymer as the first phase.

6. Material as in claim 1, wherein a solution of the second phase contains from about 1% to about 2% in weight, preferably from about 1.25% to about 1.75% in weight, of a polymer selected from among said polymers.

7. Material as in claim 1, wherein the material contains in all from about 1.75% to about 5% in weight of one of said polymers and of said reticulated hydrogel deriving from one of said polymers.

8. Material as in claim 1, wherein the first phase and the second phase are formed by particles of dried powder with a homogeneous distribution of the nominal diameter less than or equal to about 2 mm.

9. Material as in claim 1, wherein the material comprises a water or physiological solution in which the first and second phase are dispersed.

10. Product comprising a sterile confection containing the composite material as in claim 1.

11. Medical device comprising a dispersion of a desired quantity of the composite material as in claim 1 in a physiological or water solution.

12. Method to achieve a composite material suitable for biomedical use, comprising preparing a first phase comprising a reticulated hydrogel deriving from a polymer chosen from among polymers N-methyl-amides derivatives of carboxymethylcellulose, alginic acid or carboxymethyl starch, and preparing a second phase which comprises one of said polymers.

13. Method as in claim 12, further comprising preparing and purifying one of said polymers in the form of a polymer solution, preparing a dried powder of reticulated hydrogel from a part of said polymer solution and associating said dried powder of reticulated hydrogel with a dried powder obtained from another part of said polymer solution, or directly with said other part of said polymer solution.

14. Method as in claim 13, further comprising:
concentrating the polymer solution by partial dehydration,
dividing the concentrated polymer solution by partial dehydration into two fractions or parts,
totally removing the solvent from a first fraction or part,
subjecting the first fraction or part, from which the solvent has been removed, to grinding and sieving so as to obtain particles of dried powder that have a homogeneous distribution of a nominal diameter so as to obtain said first phase,
reticulating a second fraction or part of the concentrated polymer solution, obtaining the reticulated hydrogel which is subsequently purified by washing,
concentrating the reticulated hydrogel and removing the washing liquid used for purifying the hydrogel,
subjecting the concentrated reticulated hydrogel from which the washing liquid has been removed to grinding and sieving so as to obtain particles of dried powder that have a homogeneous distribution of the nominal diameter so as to obtain said second phase,
dispersing in a water or physiological solution separately one from the other the first phase and the second phase and subsequently putting together the two solutions containing the first phase and the second phase.

15. Method as in claim 14, further comprising preparing and purifying one of said polymers in the form of a polymer solution, preparing a reticulated hydrogel in solution from one part of said polymer solution and associating said reticulated hydrogel in solution with another part of said polymer solution.

16. Method as in claim 15, further comprising:
reticulating a first part of the polymer solution, obtaining the reticulated hydrogel, which is subsequently purified by washing,
removing the washing liquid used in purifying the hydrogel, by drying,
putting a second part of the polymer solution in contact with the dried hydrogel, ensuring that the polymer is incorporated inside the reticular structure of the hydrogel,
removing the solvent,
grinding the product obtained in order to obtain particles in powder with a homogeneous nominal distribution.

17. Method as in claim 13, wherein the polymer solution of one of said polymers prepared and used contains from about 1% to about 2% in weight of a selected one of said polymers.

18. Method to achieve a medical device for biomedical use which provides to disperse a desired quantity of the composite material as in claim 1, or obtainable as in claim 12, in a physiological solution or in sterile water.
